SEQUENCE LISTING (28 sequences)

SEQ ID NO: 1 - protein, 12 aa, Artificial Sequence
  Synthetic Construct
SASSSISSIYLH

SEQ ID NO: 2 - protein, 8 aa, Artificial Sequence
  Synthetic Construct
YRAKNLAS

SEQ ID NO: 3 - protein, 9 aa, Artificial Sequence
  Synthetic Construct
QQGSTIPFT

SEQ ID NO: 4 - protein, 13 aa, Artificial Sequence
  Synthetic Construct
KASGYTFTRSVMH

SEQ ID NO: 5 - protein, 17 aa, Artificial Sequence
  Synthetic Construct
YINPYNDGTKYNEKFKG

SEQ ID NO: 6 - protein, 8 aa, Artificial Sequence
  Synthetic Construct
AKSGNDGY

SEQ ID NO: 7 - protein, 108 aa, Artificial Sequence
  Synthetic Construct
EIVLTQSPTTMAASPGEKITITCSASSSISSIYLHWYQQKPGFSPKVLIYRAKNLASGVPARFSGSGSGTSYSLTIGTMEAEDVATYYCQQGSTIPFTFGSGTKLEIK

SEQ ID NO: 8 - protein, 115 aa, Artificial Sequence
  Synthetic Construct
EVQLQQSGPELVKPGASVKMSCKASGYTFTRSVMHWVKQKPGQGLEWIGYINPYNDGTKYNEKFKGKATLTSDKSSSTAYMELSSLTSEDSAVYYCAKSGNDGYWGQGTTLTVSS

SEQ ID NO: 9 - protein, 215 aa, Artificial Sequence
  Synthetic Construct
EIVLTQSPTTMAASPGEKITITCSASSSISSIYLHWYQQKPGFSPKVLIYRAKNLASGVPARFSGSGSGTSYSLTIGTMEAEDVATYYCQQGSTIPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 439 aa, Artificial Sequence
  Synthetic Construct
EVQLQQSGPELVKPGASVKMSCKASGYTFTRSVMHWVKQKPGQGLEWIGYINPYNDGTKYNEKFKGKATLTSDKSSSTAYMELSSLTSEDSAVYYCAKSGNDGYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - DNA, 645 bp, Artificial Sequence
  Synthetic Construct
gaaatcgtgctgacccagagccccaccaccatggccgccagccctggcgagaagatcaccatcacctgctccgccagcagcagcatcagctccatctacctgcactggtatcagcagaagcccggcttcagccctaaggtgctgatctaccgggccaaaaacctggccagcggcgtgcccgccagattcagcggcagcggctccggcaccagctacagcctgaccatcggcaccatggaggccgaggacgtggccacctactactgccagcagggcagcaccatccccttcaccttcggcagcggcaccaagctggagatcaagcgggctgatgcggcgcccactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 12 - DNA, 1317 bp, Artificial Sequence
  Synthetic Construct
gaagtgcagctgcagcagagcggccctgagctggtgaagcctggcgccagcgtgaagatgagctgtaaggccagcggctacaccttcaccaggagcgtgatgcactgggtgaagcagaagcccggccagggcctggagtggatcggctacatcaacccctacaacgacggcaccaagtacaacgagaagttcaagggcaaggccaccctgaccagcgacaagagcagcagcaccgcctacatggagctgtccagcctgacaagcgaggatagcgccgtgtactactgtgccaagtcgggcaatgacggctactggggccagggcaccacactgaccgtgtccagcgccaaaacgacacccccatctgtctatccgctagcccctggatctgccgcccagaccaacagcatggtgaccctgggctgtctggtgaagggctacttccctgagcctgtgacagtgacctggaacagcggctctctgtctagcggcgtgcacacattccctgccgtgctgcagagcgacctgtacaccctgagcagcagcgtgaccgtgcctagcagcacatggcctagcgagaccgtgacatgcaacgtggcccaccctgcctcttctaccaaggtggacaagaagatcgtgcccagagactgcggctgcaagccttgcatctgcaccgtgcctgaggtgagcagcgtgttcatcttcccacccaagcccaaggacgtgctcaccatcaccctcacccccaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 13 - protein, 11 aa, Artificial Sequence
  Synthetic Construct
RASQDIDNYLN

SEQ ID NO: 14 - protein, 7 aa, Artificial Sequence
  Synthetic Construct
YTSEYHS

SEQ ID NO: 15 - protein, 9 aa, Artificial Sequence
  Synthetic Construct
QQGDALPPT

SEQ ID NO: 16 - protein, 10 aa, Artificial Sequence
  Synthetic Construct
GYTFGNYWMQ

SEQ ID NO: 17 - protein, 17 aa, Artificial Sequence
  Synthetic Construct
AIYEGTGDTRYIQKFAG

SEQ ID NO: 18 - protein, 10 aa, Artificial Sequence
  Synthetic Construct
LSDYVSGFSY

SEQ ID NO: 19 - protein, 11 aa, Artificial Sequence
  Synthetic Construct
RASKDISKYLN

SEQ ID NO: 20 - protein, 7 aa, Artificial Sequence
  Synthetic Construct
YTSGYHS

SEQ ID NO: 21 - protein, 9 aa, Artificial Sequence
  Synthetic Construct
QQGDALPPT

SEQ ID NO: 22 - protein, 10 aa, Artificial Sequence
  Synthetic Construct
GYTFGNYWMQ

SEQ ID NO: 23 - protein, 17 aa, Artificial Sequence
  Synthetic Construct
AIYEGTGKTVYIQKFAD

SEQ ID NO: 24 - protein, 10 aa, Artificial Sequence
  Synthetic Construct
LSDYVSGFGY

SEQ ID NO: 25 - protein, 214 aa, Artificial Sequence
  Synthetic Construct
DIQMTQSPSSLSASVGDRVTITCRASQDIDNYLNWYQQKPGKAPKLLIYYTSEYHSGVPSRFSGSGSGTDFTFTISSLQPEDIATYYCQQGDALPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 445 aa, Artificial Sequence
  Synthetic Construct
QVQLVQSGAEVKKPGASVKVSCKASGYTFGNYWMQWVRQAPGQGLEWMGAIYEGTGDTRYIQKFAGRVTMTRDTSTSTVYMELSSLRSEDTAVYYCARLSDYVSGFSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 214 aa, Artificial Sequence
  Synthetic Construct
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 445 aa, Artificial Sequence
  Synthetic Construct
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2019/0031748 A1
(43) Pub. Date: Jan. 31, 2019

(54) CGRP ANTIBODIES AND USES THEREOF

(71) Applicant: Eli Lilly and Company, Indianapolis, IN (US)

(72) Inventors: Xiyun CHAI, Carmel, IN (US); Barrett ALLAN, Encinitas, CA (US)

(21) Appl. No.: 16/069,996

(22) PCT Filed: Jan. 20, 2017
(86) PCT No.: PCT/US2017/014325

§ 371 Date: Jul. 13, 2018

Related U.S. Application Data
(60) Provisional application No. 62/288,045, filed on Jan. 28, 2016.

(51) Int. Cl. C07K 16/18; G01N 33/74

(52) U.S. Cl. CPC C07K 16/18; G01N 33/74; C07K 2317/565; G01N 2333/585; C07K 2317/515; C07K 2317/55; C07K 2317/51

(57) ABSTRACT

The present invention relates to antibodies that bind to human CGRP, compositions and kits comprising such CGRP antibodies, and methods of using such CGRP antibodies for detection of human CGRP.

The present invention relates to antibodies that bind Calcitonin Gene-Related Peptide (CGRP) and their use in kits and methods to detect CGRP in patient samples.

CGRP is a 37 amino acid neuropeptide secreted by the nerves of the central and peripheral nervous systems. It is widely distributed in sensory nerves, both in the peripheral and central nervous systems, and displays a large number of different biological activities. When released from trigeminal and other nerve fibers, CGRP is thought to mediate its biological responses by binding to specific cell surface receptors. Elevated levels of CGRP play a role in several conditions, including migraine, cluster headache and osteoarthritis pain.

Antibodies to CGRP are well known in the art. For example, U.S. Pat. No. 8,298,536 discloses a number of anti-CGRP antibodies. Methods of measuring levels of CGRP in a patient sample are also known in the art. For example, Cayman Chemical® markets a CGRP (human) enzyme immunoassay (EIA) kit that can measure CGRP in a variety of patient samples. However, there still remains a need for antibodies and kits with higher sensitivity to measure low levels of CGRP in patient samples.

The antibodies, kits, and methods within the scope of the present invention possess the desired characteristic of high sensitivity to detect low levels of CGRP in patient samples. The present invention provides an antibody that binds to human CGRP (alpha and beta) and which comprises a light chain variable region (LCVR) and a heavy chain variable region (HCVR) wherein the LCVR comprises the amino acid sequence of SEQ ID NO: 1 at CDRL1, the amino acid sequence of SEQ ID NO: 2 at CDRL2, and the amino acid sequence of SEQ ID NO: 3 at CDRL3, and wherein the HCVR comprises the amino acid sequence of SEQ ID NO: 4 at CDRH1, the amino acid sequence of SEQ ID NO: 5 at CDRH2, and the amino acid sequence of SEQ ID NO: 6 at CDRH3. In an embodiment, the present invention provides an antibody that binds to human CGRP, comprising a light chain variable region (LCVR) having the amino acid sequence of SEQ ID NO:7, and a heavy chain variable region (HCVR) given by the amino acid sequence of SEQ ID NO:8. In a further embodiment, the present invention provides an antibody that binds to human CGRP, comprising a light chain (LC) given by the amino acid sequence of SEQ ID NO: 9, and a heavy chain (HC) given by the amino acid sequence of SEQ ID NO:10. In a more particular embodiment, the present invention provides an antibody that binds to human CGRP, comprising 2 LCs, each LC having the amino acid sequence of SEQ ID NO:9, and 2 HCs, each HC having the amino acid sequence of SEQ ID NO:10.

In another embodiment, the present invention provides a kit comprising an antibody that binds to human CGRP, comprising comprises a light chain variable region (LCVR) and a heavy chain variable region (HCVR) wherein the LCVR comprises the amino acid sequence of SEQ ID NO: 1 at CDRL1, the amino acid sequence of SEQ ID NO: 2 at CDRL2, and the amino acid sequence of SEQ ID NO: 3 at CDRL3, and wherein the HCVR comprises the amino acid sequence of SEQ ID NO: 4 at CDRH1, the amino acid sequence of SEQ ID NO: 5 at CDRH2, and the amino acid sequence of SEQ ID NO: 6 at CDRH3. In an embodiment, the present invention provides a kit comprising an antibody that binds to human CGRP, comprising a light chain variable region (LCVR) having the amino acid sequence of SEQ ID NO:7, and a heavy chain variable region (HCVR) given by the amino acid sequence of SEQ ID NO:8. In a further embodiment, the present invention provides a kit comprising an antibody that binds to human CGRP, comprising a light chain (LC) given by the amino acid sequence of SEQ ID NO: 9, and a heavy chain (HC) given by the amino acid sequence of SEQ ID NO:10. In a more particular embodiment, the present invention provides a kit comprising an antibody that binds to human CGRP, comprising 2 LCs, each LC having the amino acid sequence of SEQ ID NO:9, and 2 HCs, each HC having the amino acid sequence of SEQ ID NO:10.

In a further embodiment, the kit comprises an antibody that binds to human CGRP, comprising 2 LCs, each LC having the amino acid sequence of SEQ ID NO:9, and 2 HCs, each HC having the amino acid sequence of SEQ ID NO:10, and a second anti-CGRP antibody. More particularly, the second anti-CGRP antibody comprises a light chain variable region (LCVR) and a heavy chain variable region (HCVR) wherein the LCVR comprises the amino acid sequence of SEQ ID NO: 13 at CDRL1, the amino acid sequence of SEQ ID NO: 14 at CDRL2, and the amino acid sequence of SEQ ID NO: 15 at CDRL3, and wherein the HCVR comprises the amino acid sequence of SEQ ID NO: 16 at CDRH1, the amino acid sequence of SEQ ID NO: 17 at CDRH2, and the amino acid sequence of SEQ ID NO: 18 at CDRH3. In a more particular embodiment, the second anti-CGRP antibody comprises a light chain (LC) and a heavy chain (HC), wherein the LC comprises the amino acid sequence of SEQ ID NO: 25, and wherein the HC comprises the amino acid sequence of SEQ ID NO: 26. In another particular embodiment, the second anti-CGRP antibody comprises a light chain variable region (LCVR) and a heavy chain variable region (HCVR) wherein the LCVR comprises the amino acid sequence of SEQ ID NO: 19 at CDRL1, the amino acid sequence of SEQ ID NO:20 at CDRL2, and the amino acid sequence of SEQ ID NO: 21 at CDRL3, and wherein the HCVR comprises the amino acid sequence of SEQ ID NO: 22 at CDRH1, the amino acid sequence of SEQ ID NO: 23 at CDRH2, and the amino acid sequence of SEQ ID NO: 24 at CDRH3. In a more particular embodiment, the second anti-CGRP antibody comprises a light chain (LC) and a heavy chain (HC), wherein the LC comprises the amino acid sequence of SEQ ID NO: 27, and wherein the HC comprises the amino acid sequence of SEQ ID NO: 28.

The present invention also provides a method of detecting greater than 0.1 picogram (pg) of human CGRP per milliliter (mL) of patient sample, comprising contacting the patient sample with an antibody of the present invention. In an embodiment, the present invention provides a method of detecting 0.01-2 pg/ml of human CGRP in a patient sample, comprising contacting the patient sample with an antibody of the present invention. In another embodiment, the present invention provides a method of detecting 0.01-0.5 pg/ml of human CGRP in a patient sample, comprising contacting the patient sample with an antibody of the present invention. In a particular embodiment the present invention provides a method of detecting 0.02-0.2 pg/ml of human CGRP in a patient sample, comprising contacting the patient sample with an antibody of the present invention. In a more particular embodiment the present invention provides a method of detecting 0.02 pg/ml of human CGRP in a patient sample, comprising contacting the patient sample with an antibody of the present invention. In a particular embodiment, the patient sample is EDTA plasma, heparin plasma, serum, CSF, or synovial fluid. In another particular embodiment, the method consists of an ELISA.

The present invention also provides a method of detecting human CGRP in a patient sample comprising contacting the sample with a first anti-CGRP antibody, and detecting the amount of CGRP bound to the antibody with an antibody that comprises 2 LCs and 2 HCs, wherein the amino acid sequence of each LC is the amino acid sequence of SEQ ID NO: 9, and the amino acid sequence of each HC is the amino acid sequence of SEQ ID NO:10. In a particular embodiment, the patient sample is EDTA plasma, heparin plasma, serum, CSF, or synovial fluid. In another particular embodiment, the method consists of an ELISA. In another embodiment, the first anti-CGRP antibody comprises a light chain variable region (LCVR) and a heavy chain variable region (HCVR) wherein the LCVR comprises the amino acid sequence of SEQ ID NO: 13 at CDRL1, the amino acid sequence of SEQ ID NO: 14 at CDRL2, and the amino acid sequence of SEQ ID NO: 15 at CDRL3, and wherein the HCVR comprises the amino acid sequence of SEQ ID NO: 16 at CDRH1, the amino acid sequence of SEQ ID NO: 17 at CDRH2, and the amino acid sequence of SEQ ID NO: 18 at CDRH3. In another embodiment, the first anti-CGRP antibody comprises a light chain variable region (LCVR) and a heavy chain variable region (HCVR) wherein the LCVR comprises the amino acid sequence of SEQ ID NO: 19 at CDRL1, the amino acid sequence of SEQ ID NO: 20 at CDRL2, and the amino acid sequence of SEQ ID NO: 21 at CDRL3, and wherein the HCVR comprises the amino acid sequence of SEQ ID NO: 22 at CDRH1, the amino acid sequence of SEQ ID NO: 23 at CDRH2, and the amino acid sequence of SEQ ID NO: 24 at CDRH3.

The present invention also provides method of detecting human CGRP in a patient sample comprising contacting the sample with an antibody comprising 2 LCs and 2 HCs, wherein the amino acid sequence of each LC is the amino acid sequence of SEQ ID NO:9, and the amino acid sequence of each HC is the amino acid sequence of SEQ ID NO:10., and detecting the amount of CGRP bound to the antibody with a second anti-CGRP antibody. In a particular embodiment, the patient sample is EDTA plasma, heparin plasma, serum, CSF, or synovial fluid. In another particular embodiment, the method consists of an ELISA. In another embodiment, the second anti-CGRP antibody comprises a light chain variable region (LCVR) and a heavy chain variable region (HCVR) wherein the LCVR comprises the amino acid sequence of SEQ ID NO: 13 at CDRL1, the amino acid sequence of SEQ ID NO:14 at CDRL2, and the amino acid sequence of SEQ ID NO: 15 at CDRL3, and wherein the HCVR comprises the amino acid sequence of SEQ ID NO: 16 at CDRH1, the amino acid sequence of SEQ ID NO: 17 at CDRH2, and the amino acid sequence of SEQ ID NO: 18 at CDRH3. In another embodiment, the second anti-CGRP antibody comprises a light chain variable region (LCVR) and a heavy chain variable region (HCVR) wherein the LCVR comprises the amino acid sequence of SEQ ID NO: 19 at CDRL1, the amino acid sequence of SEQ ID NO: 20 at CDRL2, and the amino acid sequence of SEQ ID NO: 21 at CDRL3, and wherein the HCVR comprises the amino acid sequence of SEQ ID NO: 22 at CDRH1, the amino acid sequence of SEQ ID NO: 23 at CDRH2, and the amino acid sequence of SEQ ID NO: 24 at CDRH3.

The present invention also provides an antibody that binds to human CGRP and which comprises a light chain variable region (LCVR) and a heavy chain variable region (HCVR) wherein the LCVR comprises the amino acid sequence of SEQ ID NO: 1 at CDRL1, the amino acid sequence of SEQ ID NO:2 at CDRL2, and the amino acid sequence of SEQ ID NO: 3 at CDRL3, and wherein the HCVR comprises the amino acid sequence of SEQ ID NO: 4 at CDRH1, the amino acid sequence of SEQ ID NO: 5 at CDRH2, and the amino acid sequence of SEQ ID NO: 6 at CDRH3 for use in measuring the amount of CGRP in a human sample. In an embodiment, the present invention provides an antibody that binds to human CGRP, comprising a light chain variable region (LCVR) having the amino acid sequence of SEQ ID NO: 7, and a heavy chain variable region (HCVR) given by the amino acid sequence of SEQ ID NO:8 for use in measuring the amount of CGRP in a human sample. In a further embodiment, the present invention provides an antibody that binds to human CGRP, comprising a light chain (LC) given by the amino acid sequence of SEQ ID NO:9, and a heavy chain (HC) given by the amino acid sequence of SEQ ID NO:10 for use in measuring the amount of CGRP in a human sample. In a more particular embodiment, the present invention provides an antibody that binds to human CGRP, comprising 2 LCs, each LC having the amino acid sequence of SEQ ID NO:9, and 2 HCs, each HC having the amino acid sequence of SEQ ID NO:10 for use in measuring the amount of CGRP in a human sample.

In another embodiment, the present invention provides an antibody that binds to human CGRP and which comprises a light chain variable region (LCVR) and a heavy chain variable region (HCVR) wherein the LCVR comprises the amino acid sequence of SEQ ID NO: 1 at CDRL1, the amino acid sequence of SEQ ID NO:2 at CDRL2, and the amino acid sequence of SEQ ID NO: 3 at CDRL3, and wherein the HCVR comprises the amino acid sequence of SEQ ID NO: 4 at CDRH1, the amino acid sequence of SEQ ID NO: 5 at CDRH2, and the amino acid sequence of SEQ ID NO: 6 at CDRH3 for the manufacture of a detection reagent to measure CGRP in a patient sample. In an embodiment, the present invention provides an antibody that binds to human CGRP, comprising a light chain variable region (LCVR) having the amino acid sequence of SEQ ID NO: 7, and a heavy chain variable region (HCVR) given by the amino acid sequence of SEQ ID NO:8 for the manufacture of a detection reagent to measure CGRP in a patient sample. In a further embodiment, the present invention provides an antibody that binds to human CGRP, comprising a light chain (LC) given by the amino acid sequence of SEQ ID NO:9, and a heavy chain (HC) given by the amino acid sequence of SEQ ID NO:10 for the manufacture of a detection reagent to measure CGRP in a patient sample. In a more particular embodiment, the present invention provides an antibody that binds to human CGRP, comprising 2 LCs, each LC having the amino acid sequence of SEQ ID NO:9, and 2 HCs, each HC having the amino acid sequence of SEQ ID NO:10 for the manufacture of a detection reagent to measure CGRP in a patient sample.

The antibodies of the present invention bind to both alpha and beta CGRP, herein referred to as “CGRP”. As used herein, an “antibody” is an immunoglobulin molecule comprising two Heavy Chains (HC) and two Light Chains (LC) interconnected by disulfide bonds. The amino terminal portion of each LC and HC includes a variable region responsible for antigen recognition via the complementarity determining regions (CDRs) contained therein. The CDRs are interspersed with regions that are more conserved, termed framework regions (FR). There are three CDRs in each of the variable regions of the heavy chain (CDRH1, CDRH2 and CDRH3) and of the light chain (CDRL1, CDRL2, CDRL3), for each of the variable regions. Assignment of amino acids to CDR domains within the LCVR and HCVR regions of the antibodies of the present invention is based on the well-known Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)). Following the above method, the CDRs of the present invention were determined (Table 1).

“Antibody Fab fragment”, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1)single-chain Fv (scFv) molecules (2)single chain polypeptides containing only one light chain variable domain, or a fragment thereof that contains the three CDRs of the light chain variable domain, without an associated heavy chain moiety and (3)single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CHI) found in a non-Fc region of an intact antibody, and/or can contain any hinge region sequence found in an intact antibody. Preferably, the antibody fragment is a Fab fragment.

The CGRP immunoassays of the present invention can be either a competitive or sandwich-assays. At least one of the first and/or second antibodies may be labeled with a detectable label or immobilized on a solid support. . The method for labeling an antibody or antibody fragment with a detectable label is known to one ordinary skilled in the art. Examples of a detectable label include without limitation radioactive isotopes, enzymes, fluorescent substances, luminescent substances, and particles. The labeling of an antibody with a detectable label can be carried out according to a method known to one ordinary skilled in the art, for example, that described by Kono et al. (Kaku-Igaku Gijutu, 13(1), 2, (1993)).

As used herein, the term “kit” is used in reference to a combination of reagents and other materials that are required to perform an assay. It is contemplated that the kit includes at least one anti-human CGRP antibody, preferably the CA1 1 antibody of the present invention. More preferably, the kit also includes either Antibody I or Antibody II of the present invention. It is not intended that the term “kit” be limited to a particular combination of reagents and/or other materials.As used herein, the term “sandwich immunoassay” or “sandwich-assay” refers to an assay to detect antigen using a pair of antibodies (for example, antibody ‘A’ and antibody ‘B’) each directed against the antigen or a portion of the antigen. For the pair of antibodies as an example, antibody ‘A’ is labeled either covalently or non-covalently to a reporter molecule (e.g., a molecule that allows for electrochemiluminescence or a molecule that allows for fluorescence). An example of non-covalent labeling of an antibody ‘A’ would be to allow a secondary labeled antibody against the antibody ‘A’ to bind to antibody ‘A’. Antibody ‘B’ is attached directly (or allowed to attach indirectly) to a solid support phase like an assay plate, a bead, a magnet or an electrode. Detection techniques suitable for sandwich immunoassays include electrochemiluminescence, chemiluminescence, and fluorogenic chemiluminescence.

A “competitive assay” refers to unlabeled analyte in a sample that competes with labeled analyte to bind to an antibody. After washing away the unbound analyte, the amount of labeled analyte is measured.

The term “contacting” refers to bringing an antibody and the material containing the antigen together in such a manner that the antibody interacts with, or binds to, the antigen. The term “detect” or “detecting” refers to identifying the presence or existence of analyte in a sample with an unlabeled or labeled antibody.

The antibodies of the present invention are monoclonal antibodies (“mAbs”). Monoclonal antibodies can be produced, for example, by hybridoma technologies, recombinant technologies, phage display technologies, synthetic technologies, e.g., CDR-grafting, or combinations of such or other technologies known in the art. In another embodiment of the present invention, the antibody, or the nucleic acid encoding the same, is provided in isolated form. As used herein, the term “isolated” refers to a protein, peptide or nucleic acid that is not found in nature and is free or substantially free from other macromolecular species found in a cellular environment. “Substantially free”, as used herein, means the protein, peptide or nucleic acid of interest comprises more than 80% (on a molar basis) of the macromolecular species present, preferably more than 90% and more preferably more than 95%.

As used herein the term “patient” refers to a human subject. The term “sample” or patient sample are used interchangeably and as used herein, refers to a sample obtained from a patient. The sample may be of any biological tissue, cells or fluid. Such samples include, plasma (as well as EDTA or Heparin plasma), serum CSF or synovial fluid.

### EXAMPLES

### Antibody Expression and Purification

The CGRP-reactive antibody CA1 1 is expressed transiently in HEK293 cells.

The antibody is mouse IgGl/kappa and was purified using protein G. Briefly, 2 L of HEK293 supernatant from cells transfected with LC and HC vectors of mIgGl CA11 are harvested five days post transfection and loaded at 2 mL/min overnight in cold room onto a 5 mL, Protein G Column (GE Healthcare #17-0405-03). The next day, Protein G column is washed at 5 mL/min with 5 column volumes of PBS, pH 7.4. The bound CA11 antibody is eluted from Protein G column at 5 mL/min with 10 mM citric acid, pH ˜3, and immediately neutralized with 1/10 volume of 1 M Tris, pH 8. The CA11 antibody is buffer exchanged into PBS, pH 7.4, and concentrated in Millipore centrifugal concentrators to 1.7 mg/mL. Purity of the antibody is assessed using SDS-PAGE and size-exclusion chromatography. N-terminal sequencing and MALDI-TOF are used to further confirm the identity of the CA11 antibody. BIAcore™ binding is performed to determine binding affinity of CA11 antibody to CGRP peptide. Sequences of the exemplified antibodies are provided in Table 1.

TABLE 1
SEQ IDs of amino acid sequences of the exemplified antibodies.

| | Light | Heavy | | |
|---|---|---|---|---|
| Antibody | Chain | Chain | LCVR | HCVR |
| CA11 | 9 | 10 | 7 | 8 |
| Antibody I | 25 | 26 | — | — |
| Antibody II | 27 | 28 | — | — |

| Antibody | LCDR1 | LCDR2 | LCDR3 |
|---|---|---|---|
| CA11 | 1 | 2 | 3 |
| Antibody I | 13 | 14 | 15 |
| Antibody II | 19 | 20 | 21 |
| Antibody | HCDR1 | HCDR2 | HCDR3 |
| CA11 | 4 | 5 | 6 |
| Antibody I | 16 | 17 | 18 |
| Antibody II | 22 | 23 | 24 |

### CGRP Fab Binding ELISA

An ELISA based assay is used to measure the relative affinities of CA11 and parent (“C1WT”) Fab molecules. Briefly, 96 well plates are coated by adding 50 μL/well of a 1 μg/mL solution containing goat anti-human kappa (Southern Biotech #2060-01) in PBS 7.4 overnight at 4° C. Following incubation, plates are blocked with 200 μL/well of a Casein/PBS solution (Thermo-Fisher Scientific #37528) for 1 hr at room temperature. Plates are washed 3 times with PBS containing 0.1% Tween®−20 (PBS-T). Fifty μL of each Fab are normalized to a concentration of 2 μg/mL, added to separate columns on the plate, and incubated for 1 hr at 37° C. The plate is washed 3 times with PBS-T prior to incubation with 50 μL of N-terminal biotin labelled human CGRP (Abgent Custom Synthesized Peptide #355061532). Starting at 20 nM, three-fold serial dilutions of the N-terminal biotin labelled CGRP peptide are added to the captured Fabs and incubated for 1 hr at 37° C. The plate is washed 3 times with PBS-T. To facilitate dissociation of the human CGRP peptide from the captured Fabs, an additional 200 μL of PBS-T is added to each well and incubated for 2 hr at 37° C. The plate is washed, 50 μL of alkaline phosphatase conjugated neutravidin (Pierce #31002) diluted 1:1000 in PBS-T is added, and the plates are incubated for 1 hr at 37° C. The plates are washed and 50 μL of alkaline phosphatase substrate diluted 25-fold in water is added. Following colorimetric development, the plate is read using a Molecular Devices VMax Kinetic ELISA Microplate Reader and the absorbance at 560 nm is recorded as a function of human CGRP concentration, plotting with Microsoft® Excel.

TABLE 2
ELISA binding data.

| CGRP (nM) | C1WT (A560) | CA11 (A560) |
|---|---|---|
| 20 | 0.327 | 1.046 |
| 6.667 | 0.232 | 1.028 |
| 2.222 | 0.14 | 0.688 |
| 0.741 | 0.084 | 0.356 |
| 0.247 | 0.064 | 0.165 |
| 0.082 | 0.051 | 0.091 |
| 0.027 | 0.052 | 0.063 |
| 0.009 | 0.065 | 0.082 |

The data shown in Table 2 demonstrate that CA11 binds CGRP.

### High Sensitive CGRP MSD (Meso Scale Discovery®) ELISA

A Meso Scale Discovery® (MSD) assay is used to determine the ability of CA11 antibody in detecting human CGRP. Plates are blocked by adding 150 μL/well 3% Blocker A/PBS, and incubating for 60 minutes at room temperature with rotation at 650 rpm. Plates are washed 3 times with PBS-T, and diluted biotin-labelled anti-CGRP antibody (Antibody I; 0.1 μg/mL in 0.1% Blocker A/PBS) is added into wells of streptavidin plates. Plates are incubated for 1 hour at room temperature with rotation (650 rpm).

Plates are washed, 25 μ1 of human healthy donor serum, heparin plasma, CSF, or synovial fluid samples (or standard; alpha-CGRP; Bachem) are added into wells, and incubated at room temperature with rotation for 2 hours. Plates are washed, and 25 μL Sulfo-Tag labelled-anti-CGRP (CA11) (0.5 μg/mL) is added, and plates are incubated for 1 hour at room temperature with rotation. Plates are washed and 150 uL/ well 2× MSD Read Buffer T is added to each well. Plates are read on MSD instrument, and unknowns are calculated using a log-log 4-5 PL fit on the MSD Discovery Workbench software or equivalent. The CGRP concentration from human donor samples is summarized in Table 2.

To determine the spike and recovery in each of matrices, the different concentration of CGRP standard is spiked into human EDTA plasma, heparin plasma, serum, CSF or synovial fluid. The recovery of CGRP in each of matrices is summarized in Table 3.

TABLE 2
Summary of CGRP level in healthy donors.

| | | CGRP Concentration |
|---|---|---|
| Matrix | Sample Number | (pg/mL) Mean +/− SD |
| EDTA Plasma | 55 | 2.2 +/− 0.86 |
| Serum | 61 | 1.78 +/− 0.60 |
| Heparin Plasma | 10 | 1.23 +/− 1.03 |
| CSF | 20 | 5.48 +/− 1.61 |
| Synovial Fluid | 6 | 0.32 +/− 0.25 |

The data in Table 2 demonstrate that the CGRP MSD assay with the CA11 antibody detected CGRP in healthy donors.

TABLE 3
Spike and recovery of CGRP.

| | Spike | % | | Spike | % |
|---|---|---|---|---|---|
| | (pg/mL) | Recovery | | (pg/mL) | Recovery |
| EDTA | 25 | 98 | CSF | 25 | 107 |
| Plasma | 8.33 | 98 | | 8.33 | 103 |
| | 2.78 | 99 | | 2.78 | 99 |
| | 0.93 | 97 | | 0.93 | 98 |
| | 0.31 | 101 | | 0.31 | 99 |
| Serum | 25 | 83 | Synovial | 25 | 103 |
| | 8.33 | 84 | Fluid | 8.33 | 99 |
| | 2.78 | 88 | | 2.78 | 92 |
| | 0.93 | 87 | | 0.93 | 91 |
| | 0.31 | 96 | | 0.31 | 90 |
| Heparin | 30 | 107 | | | |
| Plasma | 3.3 | 112 | | | |
| | 0.37 | 115 | | | |
| | 0.12 | 108 | | | |

The data in Table 3 demonstrate that the CGRP MSD assay with the CA11 antibody detected CGRP in human EDTA plasma, serum, heparin, CSF, and synovial fluid.

### Quanterix™ Simoa™ Assay

Beads (0.5 mg/ml) are conjugated to Antibody II according to the Quanterix™ protocol. A 10 ml solution of beads (5 million beads/mL), a 10 mL solution of biotinylated CA11 antibody (0.1 μg/mL), and a 10 mL solution of streptavidin-beta-galactosidase (SBG; 150 pM) are prepared and transferred to separate 15 mL bottles. Beads, CA11 antibody, calibrators, SBG, and supplied resorufin-beta-D galactopyranoside RGP reagents are loaded into the instrument according to the Simoa™ HD-1 Analyzer User Guide. The run is initiated and run on the instrument according to the Homebrew chapter of the Simoa™ HD-1 Analyzer User Guide. Binding data is shown in Table 4.

To determine the spike and recovery in the Quanterix™ assay with the CA11 antibody, CGRP is spiked into the human plasma. The percentage of recovered spiked CGRP is summarized in Table 5.

To determine the sensitivity of the CGRP Quanterix™ assay with the CA11 antibody, CGRP levels in healthy donor plasma are detected. The concentration of CGRP detected from healthy donor plasma is shown in Table 6.

TABLE 4
CGRP Quanterix ™ Assay Binding Data.

| CGRP (pg/mL) | Average AEB* |
|---|---|
| 50 | 2.99 |
| 16.67 | 1.18 |
| 5.56 | 0.42 |
| 1.85 | 0.13 |
| 0.62 | 0.06 |
| 0.21 | 0.02 |
| 0.07 | 0.011 |
| 0.02 | 0.0075 |
| 0.0076 | 0.0079 |
| 0 | 0.0064 |

*AEB = average enzyme per bead

The data in Table 4 demonstrate that the CGRP Quanterix™ assay with the CA11 antibody detected human CGRP in plasma as low as 0.02 pg/ml.

TABLE 5
CGRP spike and recovery in EDTA plasma.

| Spike | |
|---|---|
| (pg/mL) | Recovery (%) |
| 25 | 83 |
| 8.33 | 77 |
| 2.78 | 78 |
| 0.93 | 77 |
| 0.31 | 99 |
| 0.10 | 96 |

The data in Table 5 demonstrate that the CGRP Quanterix™ assay with the CA11 antibody detected spiked CGRP in EDTA plasma with a percent recovery in the range of 77% to 99%.

TABLE 6
Plasma CGRP level in healthy donors (n = 9 donors per group).

| | CGRP Concentration |
|---|---|
| Matrix | (pg/mL) Mean +/− SD |
| EDTA Plasma | 0.93 +/− 0.64 |
| Heparin Plasma | 1.02 +/− 0.77 |

The data in Table 6 demonstrate that the CGRP Quanterix™ assay with the CA11 antibody detected about 0.93 pg/mL CGRP in healthy donor EDTA plasma, and about 1.02 pg/mL CGRP in healthy donor heparin plasma.

Sequences

SEQ ID NO: 1 (CA11 LCDR1)
SASSSISSIYLH
SEQ ID NO: 2 (CA11 LCDR2)
YRAKNLAS
SEQ ID NO: 3 (CA11 LCDR3)
QQGSTIPFT
SEQ ID NO: 4 (CA11 HCDR1)
KASGYTFTRSVMH
SEQ ID NO: 5 (CA11 HCDR2)
YINPYNDGTKYNEKFKG
SEQ ID NO: 6 (CA11 HCDR3)
AKSGNDGY
SEQ ID NO: 7 (CA11 LCVR)
EIVLTQSPTTMAASPGEKITITCSASSSISSIYLHWYQQKPGFSPKVLIYRAKNLASG
VPARFSGSGSGTSYSLTIGTMEAEDVATYYCQQGSTIPFTFGSGTKLEIK
SEQ ID NO: 8 (CA11 HCVR)
EVQLQQSGPELVKPGASVKMSCKASGYTFTRSVMHWVKQKPGQGLEWIGYINP
YNDGTKYNEKFKGKATLTSDKSSSTAYMELSSLTSEDSAVYYCAKSGNDGYWG
QGTTLTVSS
SEQ ID NO: 9 (CA11 LC)
EIVLTQSPTTMAASPGEKITITCSASSSISSIYLHWYQQKPGFSPKVLIYRAKNLASG
VPARFSGSGSGTSYSLTIGTMEAEDVATYYCQQGSTIPFTFGSGTKLEIKRADAAP
TVSIFPPSSEQLTSGGASVVCFLNNFYPKDINVKWKIDGSERQNGVLNSWTDQDS
KDSTYSMSSTLTLTKDEYERHNSYTCEATHKTSTSPIVKSFNRNEC
SEQ ID NO: 10 (CA11 HC)
EVQLQQSGPELVKPGASVKMSCKASGYTFTRSVMHWVKQKPGQGLEWIGYINP
YNDGTKYNEKFKGKATLTSDKSSSTAYMELSSLTSEDSAVYYCAKSGNDGYWG
QGTTLTVSSAKTTPPSVYPLAPGSAAQTNSMVTLGCLVKGYFPEPVTVTWNSGSL
SSGVHTFPAVLQSDLYTLSSSVTVPSSTWPSETVTCNVAHPASSTKVDKKIVPRDC
GCKPCICTVPEVSSVFIFPPKPKDVLTITLTPKVTCVVVDISKDDPEVQFSWFVDDV
EVHTAQTQPREEQFNSTFRSVSELPIMHQDWLNGKEFKCRVNSAAFPAPIEKTISK
TKGRPKAPQVYTIPPPKEQMAKDKVSLTCMITDFFPEDITVEWQWNGQPAENYK
NTQPIMDTDGSYFVYSKLNVQKSNWEAGNTFTCSVLHEGLHNHHTEKSLSHSPG
K
SEQ ID NO: 11 (CA11 LC DNA)
GAAATCGTGCTGACCCAGAGCCCCACCACCATGGCCGCCAGCCCTGGCGAGA
AGATCACCATCACCTGCTCCGCCAGCAGCAGCATCAGCTCCATCTACCTGCAC
TGGTATCAGCAGAAGCCCGGCTTCAGCCCTAAGGTGCTGATCTACCGGGCCA
AAAACCTGGCCAGCGGCGTGCCCGCCAGATTCAGCGGCAGCGGCTCCGGCAC
CAGCTACAGCCTGACCATCGGCACCATGGAGGCCGAGGACGTGGCCACCTAC
TACTGCCAGCAGGGCAGCACCATCCCCTTCACCTTCGGCAGCGGCACCAAGC
TGGAGATCAAGCGGGCTGATGCGGCGCCCACTGTATCCATCTTCCCACCATCC
AGTGAGCAGTTAACATCTGGAGGTGCCTCAGTCGTGTGCTTCTTGAACAACTT
CTACCCCAAAGACATCAATGTCAAGTGGAAGATTGATGGCAGTGAACGACAA
AATGGCGTCCTGAACAGTTGGACTGATCAGGACAGCAAAGACAGCACCTACA
GCATGAGCAGCACCCTCACGTTGACCAAGGACGAGTATGAACGACATAACAG
CTATACCTGTGAGGCCACTCACAAGACATCAACTTCACCCATTGTCAAGAGCT
TCAACAGGAATGAGTGT
SEQ ID NO: 12 (CA11 HC DNA)
GAAGTGCAGCTGCAGCAGAGCGGCCCTGAGCTGGTGAAGCCTGGCGCCAGCG
TGAAGATGAGCTGTAAGGCCAGCGGCTACACCTTCACCAGGAGCGTGATGCA
CTGGGTGAAGCAGAAGCCCGGCCAGGGCCTGGAGTGGATCGGCTACATCAAC
CCCTACAACGACGGCACCAAGTACAACGAGAAGTTCAAGGGCAAGGCCACCC
TGACCAGCGACAAGAGCAGCAGCACCGCCTACATGGAGCTGTCCAGCCTGAC
AAGCGAGGATAGCGCCGTGTACTACTGTGCCAAGTCGGGCAATGACGGCTAC
TGGGGCCAGGGCACCACACTGACCGTGTCCAGCGCCAAAACGACACCCCCAT
CTGTCTATCCGCTAGCCCCTGGATCTGCCGCCCAGACCAACAGCATGGTGACC
CTGGGCTGTCTGGTGAAGGGCTACTTCCCTGAGCCTGTGACAGTGACCTGGAA
CAGCGGCTCTCTGTCTAGCGGCGTGCACACATTCCCTGCCGTGCTGCAGAGCG
ACCTGTACACCCTGAGCAGCAGCGTGACCGTGCCTAGCAGCACATGGCCTAG
CGAGACCGTGACATGCAACGTGGCCCACCCTGCCTCTTCTACCAAGGTGGAC
AAGAAGATCGTGCCCAGAGACTGCGGCTGCAAGCCTTGCATCTGCACCGTGC
CTGAGGTGAGCAGCGTGTTCATCTTCCCACCCAAGCCCAAGGACGTGCTCACC
ATCACCCTCACCCCCAAGGTCACGTGTGTTGTGGTAGACATCAGCAAGGATG
ATCCCGAGGTCCAGTTCAGCTGGTTTGTAGATGATGTGGAGGTGCACACAGCT
CAGACGCAACCCCGGGAGGAGCAGTTCAACAGCACTTTCCGCTCAGTCAGTG
AACTTCCCATCATGCACCAGGACTGGCTCAATGGCAAGGAGTTCAAATGCAG
GGTCAACAGTGCAGCTTTCCCTGCCCCCATCGAGAAAACCATCTCCAAAACC
AAAGGCAGACCGAAGGCTCCACAGGTGTACACCATTCCACCTCCCAAGGAGC
AGATGGCCAAGGATAAAGTCAGTCTGACCTGCATGATAACAGACTTCTTCCCT
GAAGACATTACTGTGGAGTGGCAGTGGAATGGGCAGCCAGCGGAGAACTAC
AAGAACACTCAGCCCATCATGGACACAGATGGCTCTTACTTCGTCTACAGCA
AGCTCAATGTGCAGAAGAGCAACTGGGAGGCAGGAAATACTTTCACCTGCTC
TGTGTTACATGAGGGCCTGCACAACCACCATACTGAGAAGAGCCTCTCCCACT
CTCCTGGTAAA
SEQ ID NO: 13 (Antibody I LCDR1)
RASQDIDNYLN
SEQ ID NO: 14 (Antibody I LCDR2)
YTSEYHS
SEQ ID NO: 15 (Antibody I LCDR3)
QQGDALPPT
SEQ ID NO: 16 (Antibody I HCDR1)
GYTFGNYWMQ
SEQ ID NO: 17 (Antibody I HCDR2)
AIYEGTGDTRYIQKFAG
SEQ ID NO: 18 (Antibody I HCDR3)
LSDYVSGFSY
SEQ ID NO: 19 (Antibody II LCDR1)
RASKDISKYLN
SEQ ID NO: 20 (Antibody II LCDR2)
YTSGYHS
SEQ ID NO: 21 (Antibody II LCDR3)
QQGDALPPT
SEQ ID NO: 22 (Antibody II HCDR1)
GYTFGNYWMQ
SEQ ID NO: 23 (Antibody II HCDR2)
AIYEGTGKTVYIQKFAD
SEQ ID NO: 24 (Antibody II HCDR3)
LSDYVSGFGY
SEQ ID NO: 25 (Antibody I LC)
DIQMTQSPSSLSASVGDRVTITCRASQDIDNYLNWYQQKPGKAPKLLIYYTSEYH
SGVPSRFSGSGSGTDFTFTISSLQPEDIATYYCQQGDALPPTFGQGTKLEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 26 (Antibody I HC)
QVQLVQSGAEVKKPGASVKVSCKASGYTFGNYWMQWVRQAPGQGLEWMGAI
YEGTGDTRYIQKFAGRVTMTRDTSTSTVYMELSSLRSEDTAVYYCARLSDYVSG
FSYWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDK
RVESKYGPPCPPCPAPEAAGGPSVFLFPPKPKDTLMISRTPEVTCVVVDVSQEDPE
VQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHN
HYTQKSLSLSLG
SEQ ID NO: 27 (Antibody II LC)
DIQMTQSPSSLSASVGDRVTITCRASKDISKYLNWYQQKPGKAPKLLIYYTSGYH
SGVPSRFSGSGSGTDFTLTISSLQPEDFATYYCQQGDALPPTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 28 (Antibody II HC)
QVQLVQSGAEVKKPGSSVKVSCKASGYTFGNYWMQWVRQAPGQGLEWMGAI
YEGTGKTVYIQKFADRVTITADKSTSTAYMELSSLRSEDTAVYYCARLSDYVSGF
GYWGQGTTVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKR
VESKYGPPCPPCPAPEAAGGPSVFLFPPKPKDTLMISRTPEVTCVVVDVSQEDPEV
QFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNH
YTQKSLSLSLG

We claim:

1. An antibody or antibody fragment thereof that binds to human CGRP and comprises a light chain variable region (LCVR) and a heavy chain variable region (HCVR), wherein the LCVR comprises the amino acid sequence of SEQ ID NO: 1 at CDRL1, the amino acid sequence of SEQ ID NO: 2 at CDRL2, and the amino acid sequence of SEQ ID NO: 3 at CDRL3, and wherein the HCVR comprises the amino acid sequence of SEQ ID NO: 4 at CDRH1, the amino acid sequence of SEQ ID NO: 5 at CDRH2, and the amino acid sequence of SEQ ID NO: 6 at CDRH3.

2. The antibody or antibody fragment of claim 1, wherein the LCVR has the amino acid sequence of SEQ ID NO:7 and the HCVR has the amino acid sequence of SEQ ID NO: 8.

3. The antibody of claim 1, further comprising a light chain (LC) and a heavy chain (HC), wherein the LC has the amino acid sequence of SEQ ID NO: 9 and the HC has the amino acid sequence of SEQ ID NO: 10.

4. The antibody of claim 3, wherein the antibody comprises two LCs and two HCs, wherein each LC has the amino acid sequence of SEQ ID NO: 9, and each HC has the amino acid sequence of SEQ ID NO: 10.

5. The antibody fragment of claim 1, wherein the antibody fragment is an antibody Fab fragment.

6. The antibody or antibody fragment according to claim 1, wherein the antibody or antibody fragment is labelled with a detectable label.

7. A kit comprising a first CGRP antibody comprising a light chain variable region (LCVR) and a heavy chain variable region (HCVR), wherein the LCVR comprises the amino acid sequence of SEQ ID NO: 1 at CDRL1, the amino acid sequence of SEQ ID NO: 2 at CDRL2, and the amino acid sequence of SEQ ID NO: 3 at CDRL3, and wherein the HCVR comprises the amino acid sequence of SEQ ID NO: 4 at CDRH1, the amino acid sequence of SEQ ID NO: 5 at CDRH2, and the amino acid sequence of SEQ ID NO: 6 at CDRH3 and a second CGRP antibody.

8. (canceled)

9. The kit of claim 7, wherein the second CGRP antibody comprises a light chain variable region (LCVR) and a heavy chain variable region (HCVR) wherein the LCVR comprises the amino acid sequence of SEQ ID NO: 13 at CDRL1, the amino acid sequence of SEQ ID NO: 14 at CDRL2, and the amino acid sequence of SEQ ID NO: 15 at CDRL3, and wherein the HCVR comprises the amino acid sequence of SEQ ID NO: 16 at CDRH1, the amino acid sequence of SEQ ID NO: 17 at CDRH2, and the amino acid sequence of SEQ ID NO: 18 at CDRH3.

10. The kit of claim 7, wherein the second CGRP antibody comprises a light chain variable region (LCVR) and a heavy chain variable region (HCVR) wherein the LCVR comprises the amino acid sequence of SEQ ID NO: 19 at CDRL1, the amino acid sequence of SEQ ID NO: 20 at CDRL2, and the amino acid sequence of SEQ ID NO: 21 at CDRL3, and wherein the HCVR comprises the amino acid sequence of SEQ ID NO: 22 at CDRH1, the amino acid sequence of SEQ ID NO: 23 at CDRH2, and the amino acid sequence of SEQ ID NO: 24 at CDRH3.

11. A method of detecting human CGRP in a patient sample comprising contacting the patient sample with a first CGRP antibody, and second CGRP antibody comprising a light chain variable region (LCVR) and a heavy chain variable region (HCVR), wherein the LCVR comprises the amino acid sequence of SEQ ID NO: 1 at CDRL1, the amino acid sequence of SEQ ID NO: 2 at CDRL2, and the amino acid sequence of SEQ ID NO: 3 at CDRL3, and wherein the HCVR comprises the amino acid sequence of SEQ ID NO: 4 at CDRH1, the amino acid sequence of SEQ ID NO: 5 at CDRH2, and the amino acid sequence of SEQ ID NO: 6 at CDRH3 and measuring the amount of the first or second antibody that is bound to the human CGRP in order to determine the amount of human CGRP in the patient sample.

12. The method of claim 11, wherein the patient sample comprises plasma, EDTA plasma, heparin plasma, serum, CSF, or synovial fluid.

13. (canceled)

14. The method of claim 11, wherein the first CGRP antibody comprises a light chain variable region (LCVR) and a heavy chain variable region (HCVR) wherein the LCVR comprises the amino acid sequence of SEQ ID NO: 13 at CDRL1, the amino acid sequence of SEQ ID NO: 14 at CDRL2, and the amino acid sequence of SEQ ID NO: 15 at CDRL3, and wherein the HCVR comprises the amino acid sequence of SEQ ID NO: 16 at CDRH1, the amino acid sequence of SEQ ID NO: 17 at CDRH2, and the amino acid sequence of SEQ ID NO: 18 at CDRH3.

15. The method of claim 11, wherein the first CGRP antibody comprises a light chain variable region (LCVR) and a heavy chain variable region (HCVR), wherein the LCVR comprises the amino acid sequence of SEQ ID NO: 19 at CDRL1, the amino acid sequence of SEQ ID NO: 20 at CDRL2, and the amino acid sequence of SEQ ID NO: 21 at CDRL3, and wherein the HCVR comprises the amino acid sequence of SEQ ID NO: 22 at CDRH1, the amino acid sequence of SEQ ID NO: 23 at CDRH2, and the amino acid sequence of SEQ ID NO: 24 at CDRH3.

16. The method of claim 11, wherein the first CGRP antibody is labeled with a detectable label.

17. The method of claim 11, wherein the second CGRP antibody is labeled with a detectable label.

18. (canceled)

19. (canceled)

20. (canceled)

21. (canceled)

22. (canceled)

23. (canceled)

24. (canceled)

25. (canceled)

26. (canceled)

27. (canceled)

28. (canceled)
